(19) United States
(12) Patent Application Publication
(10) Pub. No.: US 2016/0038029 A1
(43) Pub. Date: Feb. 11, 2016

(54) SYSTEM AND METHOD FOR FLUORESCENCE TOMOGRAPHY

(71) Applicant: BOARD OF REGENTS OF THE UNIVERSITY OF TEXAS SYSTEM, Austin, TX (US)

(72) Inventors: Chinmay DARNE, Houston, TX (US); Yujie LU, Houston, TX (US); I-Chih TAN, Houston, TX (US); Banghe ZHU, Houston, TX (US); Eva SEVICK-MURACA, Houston, TX (US)

(21) Appl. No.: 14/777,195

(22) PCT Filed: Mar. 17, 2014
(86) PCT No.: PCT/US14/30264

§ 371 Date: Sep. 15, 2015

Related U.S. Application Data
(60) Provisional application No. 61/787,660, filed on Mar. 15, 2013.

(51) Int. Cl. A61B 5/00; A61G 13/10; A61B 6/00; A61B 6/03; A61B 6/04

(52) U.S. Cl. CPC A61B 5/0073; A61B 5/0071; A61B 6/032; A61B 6/0407; A61B 6/583; A61G 13/10; A61B 2562/0242

(57) ABSTRACT

Systems and methods for near-infrared fluorescence (NIRF) imaging and frequency-domain photon migration (FDPM) measurements. An optical tomography system includes a bed, a wheel, a light source, an image detector, and radio frequency (RF) circuitry. The bed is configured to support an object to be imaged. The wheel is configured to rotate about the bed. The light source is coupled to the wheel. The image detector is coupled to the wheel and disposed to capture images of the object. The RF circuitry is coupled to the light source and the image detector. The RF circuitry is configured to simultaneously generate a modulation signal to modulate the light source, and generate a demodulation signal to modulate a gain of the image detector.

### CROSS REFERENCE TO RELATED APPLICATIONS

This application is a 35 U.S.C. §371 national stage application of PCT/US2014/030264 filed Mar. 17, 2014, and entitled “System and Method for Fluorescence Tomography,” which claims benefit of provisional application Ser. No. 61/787,660, filed on Mar. 15, 2013, entitled “System and Method for Fluorescence Tomography,” each of which is hereby incorporated herein by reference in its entirety.

### STATEMENT REGARDING FEDERALLY SPONSORED RESEARCH OR DEVELOPMENT

This invention was made with U.S. Government support under CA135673 awarded by the National Institutes of Health. The government has certain rights in the invention.

### BACKGROUND

Hybrid tomography systems that provide anatomical information through computed tomography (CT) or magnetic resonance (MR) and molecular biochemical information through positron emission tomography (PET) or single-photon emission computed tomography (SPECT) can accelerate pre-clinical discovery by offering features of quantitative imaging and by enabling direct translation of discoveries via clinical imaging systems. As bioanalytical techniques have migrated from assays using radionuclides to assays employing fluorescence and bioluminescence, imaging systems (e.g., small animal imaging systems) have likewise begun to transition from planar to tomographic fluorescence and bioluminescence techniques. In addition, planar near-infrared fluorescence (NIRF) has been successfully translated into humans using microdoses of contrast agent.

Recent work in pre-clinical imaging increasingly incorporates optical imaging with other complementary techniques including combinations such as bioluminescence-PET imaging-CT/MRI, fluorescence-CT, fluorescence-MRI imaging, and bioluminescence-diffuse optical tomography (DOT)-CT for acquiring and interpreting meaningful functional images. While fluorescence, bioluminescence, and nuclear imaging techniques provide excellent molecular sensitivity, CT and MRI provide high resolution and accurate anatomical referencing along with surface boundary delineation. However for time-independent fluorescence and bioluminescence techniques, heterogeneous optical properties associated with different tissue structures and organs can attenuate light, confounding image reconstruction. In vivo fluorescence reconstruction can be improved upon by incorporating segmented CT images as a priori information for use in optical reconstruction algorithms. Structural a priori information in the form of a CT derived anatomical map along with functional a priori information using scattering and absorption coefficients estimated from diffusion optical tomography (DOT) can further be utilized to enhance the imaging performance. Although CT and MRI may provide complementary anatomical tissue information, neither offers contrast that is dependent directly on tissue optical properties that impacts optical imaging.

Nuclear, bioluminescence, and fluorescence techniques provide functional information arising from probes with different emission energies. Simultaneous PET-fluorescence imaging using a conical shaped mirror placed within the PET scanner to generate separate FDG-PET scans and IRDye800-DG 3D fluorescence tomographs from an animal in a rotation-free gantry has been implemented. Interferometry provided surface information for fluorescence image reconstruction, and although not identical, the two scans employing different modalities and different imaging agents with varied pharmacokinetics showed some similarities. Using phantoms, SPECT priors have been utilized for suppressing reconstruction related artifacts and for achieving quicker solution to the fluorescence inverse imaging problem. Improvements have been made by integrating non-contact fluorescence and gamma collection within the same rotating gantry capable of projections over 360°.

### SUMMARY

Systems and methods for near-infrared fluorescence (NIRF) imaging using time-independent continuous wave (CW) and/or frequency-domain photon migration (FDPM) measurements are disclosed herein. In one embodiment, an optical tomography system includes a bed, a wheel, a light source, an image detector, and radio frequency (RF) circuitry. The bed is configured to support an object to be imaged. The wheel is configured to rotate about the bed. The light source is coupled to the wheel. The image detector is coupled to the wheel and disposed to capture images of the object. The RF circuitry is coupled to the light source and the image detector. The RF circuitry is configured to simultaneously generate a modulation signal to modulate the light source, and generate a demodulation signal to modulate a gain of the image detector. In CW mode, the RF power is zero and the light source and image detector are not modulated.

The RF circuitry may include an oscillator configured to generate an oscillation signal, and a splitter coupled to an output of the oscillator. The splitter is configured to provide the oscillation signal to the light source and the image detector. The RF circuitry may also include a phase shifter coupled to the splitter and the image detector. The phase shifter is configured to selectably vary the phase of the oscillation signal provided to the image detector with respect to the light source. A bias circuit may be coupled to the light source. The bias circuit is configured to superimpose the oscillation signal on a bias voltage that drives the light source.

The image detector may include a camera coupled to an image intensifier configured to intensify detected fluorescent light, or may include a high sensitivity camera that supports gain modulation at a high frequency. The demodulation signal modulates the gain of the image detector. A bias circuit may be coupled to the image detector. The bias circuit is configured to superimpose the oscillation signal on a bias voltage that drives the gain of the image detector.

A computerized tomography scanner may be coupled to the wheel.

A plurality of optical filters may be disposed on the wheel between the bed and the image detector.

A motorized mount may couple the image detector to the wheel. The motorized mount is configured to vary the distance between the image detector and the object.

The system may also include a control and image processing system configured to generate a transformation matrix to map frequency domain photon migration (FDPM) and continuous wave (CW) measurements generated from images acquired by the image detector to computerized tomography (CT) scans that define the boundary surface; wherein the matrix is generated by relating spatial coordinates of a phantom surface collected using optical measurements (FDPM or CW) to spatial coordinates derived from CT scans of the phantom surface; and to apply the transformation matrix to generate a boundary position of FDPM measurements for use in generating an interior image of fluorescence.

In another embodiment, a method for performing frequency domain photon migration (FDPM) and continuous wave (CW) measurements in an optical tomography system includes generating, by the optical tomography system, a transformation matrix that allows determination of surface location of fluorescence measurements and excitation illumination locations using a calibration phantom to determine boundary surface locations defined by a computerized tomography (CT) scanner. An object to be imaged is positioned in a path between a light source and an image detector. The object is illuminated with the light source from a plurality of angles, and fluorescence data produced by the object responsive to the illuminating is captured. The object is irradiated with X-rays, and the X-rays are captured to generate a CT scan of the boundary surface. The transformation matrix is applied to align captured fluorescence data with surface locations that correspond to the surfaces acquired from the CT scan. A composite image comprising the aligned fluorescence data and the CT scan image is generated.

The method may also include acquiring a baseline measurement of phase delay in optical signal generation and capture paths of the optical tomography system by measuring emission of a modulated light source via an image detector comprising a modulated gain; positioning a calibration phantom in a path between the light source and the image detector; and defining a surface of the calibration phantom relative to the light source and relative to the (CT) scanner to generate the transformation matrix. In some embodiments, a position of a galvanometer mirror to illuminate different surface locations on the calibration phantom may be determined.

The method may also include modulating the light source with a bias voltage applied to a radio frequency (RF) oscillating signal, and modulating gain of the image detector with a bias voltage applied to an RF oscillating signal. The oscillating signal applied to the image detector may be phase shifted relative to the oscillating signal applied to the light source by up to 360 degrees as the object is illuminated by the light source at the plurality of angles.

Illuminating the object with the light source may include rotating a wheel to which the light source and image detector are attached to position the light source and image detector at the plurality of angles.

In a further embodiment, an optical tomography system includes a bed, a plurality of light sources, a plurality of image detectors, and radio frequency (RF) circuitry. The bed is configured to support an object to be imaged. The plurality of light sources are disposed about the bed and configured to illuminate the object from different angles. The plurality of image detectors are disposed about the bed and configured to capture images of the object as the object is illuminated by the light sources. The RF circuitry is coupled to the light sources and detectors. The RF circuitry configured to modulate the light sources, and to demodulate image signals detected by the image detectors.

The RF circuitry may include an oscillator configured to generate an oscillation signal, and a splitter coupled to an output of the oscillator. The splitter is configured to provide the oscillation signal to the light sources and the image detectors. The RF circuitry may also include a phase shifter coupled to the splitter and the image detectors. The phase shifter is configured to selectably vary the phase of the oscillation signal provided to the image detectors with respect to the light sources. Each of the image detectors may include a camera coupled to an image intensifier configured to intensify detected light. The phase varied oscillation signal provided to the image detectors modulates a gain of the image detector.

### BRIEF DESCRIPTION OF THE DRAWINGS

For a detailed description of exemplary embodiments of the invention, reference will now be made to the accompanying drawings in which:

FIG. 1 shows a block diagram of a near-infrared fluorescence (NIRF) imaging system used for CW and frequency-domain photon migration (FDPM) measurements in accordance with various embodiments;

FIG. 2A shows RF circuitry within an enclosure for mounting on a wheel in accordance with various embodiments;

FIG. 2B shows components of a NIRF FDPM system disposed within a gantry in accordance with various embodiments;

FIG. 3 shows a schematic of the phantom employed to generate transformation matrix mapping 2-D FDPM measurements onto a 3-D surface in accordance with various embodiments;

FIG. 4 shows a flow diagram for performing FDPM-based measurements within the gantry;

FIG. 5 shows tomographic reconstructions using a bench top FDPM system and a gantry installed FDPM system in accordance with various embodiments;

FIGS. 6A and 6B show multimodal reconstructed results including fluorescence gene reporter tomography (FGRT) imaging in accordance with various embodiments; and

FIG. 7 shows a schematic diagram of a NIRF imaging system used for CW and FDPM measurements in accordance with various embodiments.

### NOTATION AND NOMENCLATURE

Certain terms are used throughout the following description and claims to refer to particular system components. As one skilled in the art will appreciate, companies may refer to a component by different names. This document does not intend to distinguish between components that differ in name but not function. In the following discussion and in the claims, the terms “including” and “comprising” are used in an open-ended fashion, and thus should be interpreted to mean “including, but not limited to . . . . ” Also, the term “couple” or “couples” is intended to mean either an indirect or direct connection. Thus, if a first device couples to a second device, that connection may be through a direct connection, or through an indirect connection via other devices and connections. The recitation “based on” is intended to mean “based at least in part on.” Therefore, if X is based on Y, X may be based on Y and any number of other factors.

As used herein, “subject” or “animal” includes both human and non-human animals. The term “non-human animals” as used in the present disclosure includes all vertebrates, e.g., mammals and non-mammals, including but not limited to laboratory animals (e.g., non-human primates, rodents), companion animals and livestock e.g., sheep, cat, dog, cow, chickens, amphibians, reptiles, etc.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Instrumentation for different imaging techniques such as computed tomography (CT), positron emission tomography (PET), single-photon emission computed tomography (SPECT), and near-infrared fluorescence (NIRF) can be efficiently designed and operated to work as stand-alone modalities. Suitable co-registration approaches, such as use of fiducial markers, may be used to co-register the data acquired from all modalities. However, combining these distinct modalities into a single gantry-based system offers advantages such as (i) optimizing cost by housing multiple electronics together and reduced instrumentation space, (ii) enabling a one-time calibration to generate transformation matrix that allows precise and repeatable data co-registration between different modalities, and (iii) automating and speeding-up entire imaging session by minimizing the time associated with animal handling. The reduced footprints of optical devices, such as diodes, photomultiplier tubes (PMTs), avalanche photodiodes (APDs), and fiber couplers permit integration of hybrid modalities within the gantries of small animal scanners or other scanners.

Despite the progress in the area, the validation of quantitative fluorescence tomography results remain to be performed with respect to the conventional PET or SPECT standardized uptake value (SUV) units. Current validation limitations include: (i) the continuing need for dual-labeled agents for direct correlation of quantitative nuclear and fluorescence imaging modalities, (ii) the confounding effects of heterogeneous tissue optical properties on intensity-based or continuous wave (CW) measurements, and (iii) the space limitations of the bulky components necessary for time-dependent measurement techniques such as time-domain photon migration (TDPM), based on pulsed laser excitation, or frequency-domain photon migration (FDPM) techniques, based on intensity modulated excitation. Although PMTs and APDs can provide a small footprint for fluorescence detection, they lack the integrating capacity of the bulkier intensified charge-coupled devices (ICCD). TDPM and FDPM techniques may be less sensitive to variations in heterogeneous optical properties within the tissue because of the added contrast provided by the fluorescence lifetime of a fluorophore. TDPM approaches require time consuming single photon counting, thus FDPM approaches may be best suited for rapid quantitative tomography. Yet the large equipment footprint associated with FDPM methods has been largely prohibitive for direct integration into the gantries of conventional imaging modalities.

Embodiments of the present disclosure include a novel miniaturized FDPM fluorescence tomography system suitable for incorporation into a scanner, such as a small animal microPET/CT scanner or other scanner. Incorporation of the optical system within the scanner enables (i) automated and controlled animal bed movement prompting quicker image acquisitions and data co-registration between the three modalities, (ii) use of CT to define volumetric meshes for fluorescence tomographic image reconstruction, and, (iii) comparison of the reconstructed values of fluorescent probe uptake with % ID/gm of radio-labeled agents for validation of quantitative imaging. The FDPM tomography system uses a contact-free excitation source and a gain-modulated, NIR-sensitive ICCD camera for homodyne detection of fluorescent phase and amplitude.

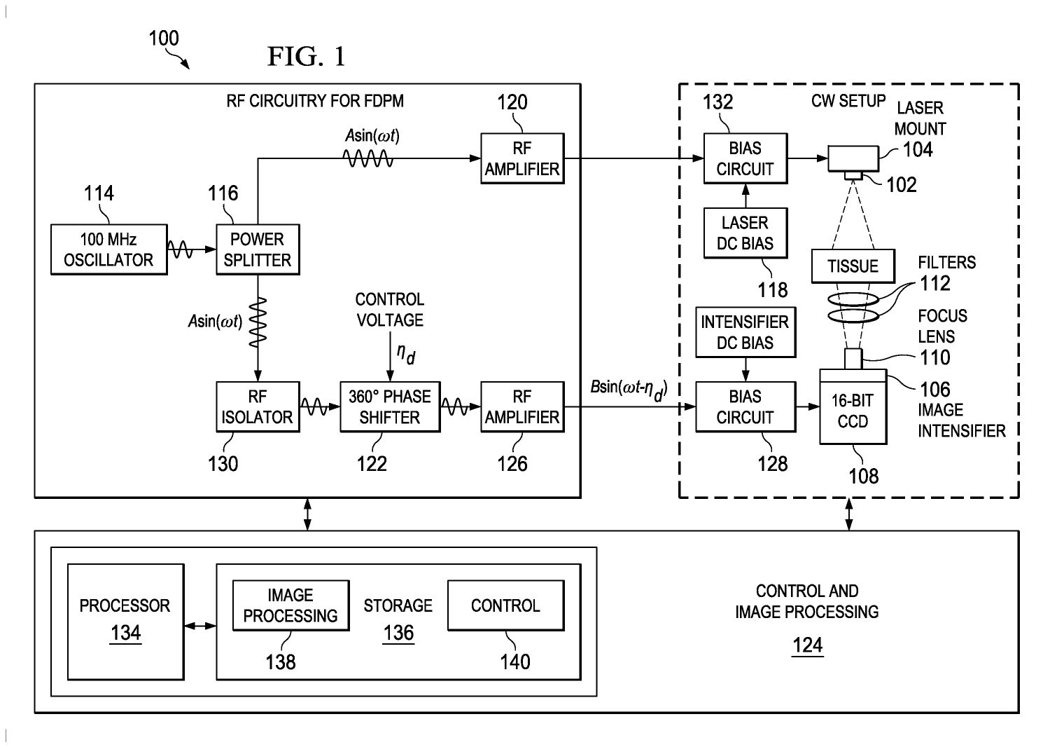

FIG. 1 shows a block diagram of a NIRF imaging system 100 used for CW and FDPM measurements in accordance with various embodiments. The CW components of the system 100 include a laser diode 102, a laser diode mount 104, diode driver (not shown), and temperature controller (not shown). The laser diode 102 provides excitation light and may be, for example, a 500 mW 785 nm diode, such as 1005-9MM-78503, by Intense Inc., North Brunswick, N.J. The laser diode mount 104, diode driver, and temperature controller may be TCLDM9, LDC205, and TED200 respectively, by Thorlabs, Newton, N.J. In some embodiments, the laser diode 102 may be replaced by a different light source.

The system 100 also includes an aspheric lens and a bandpass filter. The aspheric lens is used to collimate the laser beam, and may be a C240TME-B by Thorlabs, Newton, N.J. The bandpass filter reduces light emanating from the “side-band” wavelengths and thus minimizes the background noise from backscattered light. The bandpass filter may be a 785±10 nm bandpass filter such as LD01-785/10-12.5 by, Semrock Inc., Rochester, N.Y.

To collect a fluorescence signal emanating from the tissue illuminated by the laser diode 102, the system 100 includes a near infrared (NIR) sensitive Gen III image intensifier 106 optically coupled to a 16-bit, frame-transfer CCD camera 108. The image intensifier 106 and the CCD camera 108 form an intensifier-charge-coupled device (ICCD) detector. While using a highly-sensitive camera that is capable of modulating at RF frequency of 100 MHz or higher makes the use of intensifier redundant, in the absence of such a commercially available camera embodiments make the use of the intensifier 106 for NIRF imaging. In the rest of the disclosure the combination of intensifier 106 and CCD camera 108, for the present case, or a highly-sensitive camera, when available, are be referred to as an imaging detector or an image detector. The image intensifier 106 may be an FS9910C, by ITT EXELIS, Roanoke, Va. The CCD camera 108 may include a 1024×1024 pixel area. Such cameras as manufactured by Princeton Instruments, Trenton, N.J. may be suitable. In some embodiments, a CCD camera 108 may be repackaged into a smaller unit for integration into a gantry. For example, repackaging may reduce the original camera size from about 12 pounds (lbs) and ˜2040 cubic centimeters (cm³) to about 2 lbs and ˜820 cm³.

The system 100 includes a high voltage power supply. For CW measurements, the high voltage power supply provides the intensifier photocathode with a DC voltage of −250 V, the multichannel plate (MCP) with a variable gain between 0-1000 V (referred to herein as the intensifier gain), and the output phosphor screen with 4000 V. The high voltage power supply may be a PS20060500 by GBS Micro Power Supply, San Jose, Calif.

The system 100 includes a 28 mm wide angle lens 110 attached to the front-end of the CCD detector assembly. The lens 110 allows collection of a focused image, and may be a Nikkor f2.8D AF by Nikon. The system 100 may further include suitable 830 nm bandpass filter combinations 112 (e.g., 10±2 nm bandwidth and optical density (OD)>5 outside the passing band) to efficiently collect the weak emission signals while reducing the excitation light leakage and thus improving the image quality. The filters can be placed in different combinations depending on the degree of excitation light rejection and collection of emission signal such as (i) both placed in front of the focus lens, (ii) one filter placed before and other after the focus lens, and (iii) both placed behind the focus lens.

The system 100 includes radio frequency (RF) circuitry for time-dependent measurements used in frequency-domain photon migration (FDPM) approach. Unlike conventional systems which use two phase-locked RF oscillators, embodiments of the system 100 include a fixed frequency, RF oscillator 114 (e.g., PL0X100-10, Luff Research, Floral Park, N.Y.) and a two-way power splitter 116 (e.g., ZX-10-2-12, Mini-Circuits, Brooklyn, N.Y.) for simultaneous modulation of the laser diode 102 and demodulation of the detected signal at the imaging detector. In some embodiments, the oscillator 114 may be a 100 MHz oscillator. Other embodiments of the oscillator 114 may provide a different oscillation frequency or a range of frequencies. This homodyne configuration produces a steady-state image at the intensifier phosphor screen that is efficiently captured through integration of the CCD array 108 to maximize the signal-to-noise ratio (SNR). Laser modulation is accomplished using a bias circuit (tee) 132 to superimpose the RF signal from the RF amplifier 120 onto the laser diode DC bias 118. The DC bias 118 and the RF signal strength are selected to maximize the source modulation depth (AC/DC ratio) in order to enhance SNR and hence improve the measurement precision. A power level of +22 dBm to the laser diode 102 is achieved by amplifying the RF output from the power-splitter 116 using an RF amplifier 120 (e.g., a 5W amplifier, such as ZHL-03-5WF, Mini-Circuits, Brooklyn, N.Y.).

The system 100 includes an analog phase-shifter 122 (e.g., 9520-37, Emhiser Tele-Tech Inc., Belgrade, Mont.) to introduce a series of phase-delays from 0° to 360° to the RF signal modulating the imaging detector with respect to the laser diode signal. The non-linear behavior of the phase-shifter 122 in response to the control voltage is calibrated and programmed into a controller. The controller may be a computer executing LABVIEW (National Instruments, Austin, Tex.) to control the phase-delay. The controller may be embodied in the control and image processing subsystem 124 that controls the optical signal generation and detection, and provides processing of images acquired by the CCD camera 108 or imaging detector.

To prevent transient noise to laser diode control, the system 100 includes a unidirectional RF isolator 130 (e.g., RFLC-HXD-7A, RF-Lambda Inc., Plano, Tex.) to isolate any reflected RF signals generated by impedance mismatch from feeding back to the laser diode or source. Coaxial attenuators (VAT-X+, Mini-Circuits, Brooklyn, N.Y.) are disposed between different circuit components to dissipate excess RF power and ensure that the required power levels are delivered to the devices. The output of the phase-shifter (+6 dBm) is amplified to +40 dBm using an RF amplifier 126 (e.g., a 20 W amplifier, ZHL-20W-13, Mini-Circuits, Brooklyn, N.Y.). This amplified RF signal is superimposed onto −36 V DC bias of intensifier photocathode via a biasing circuit 128.

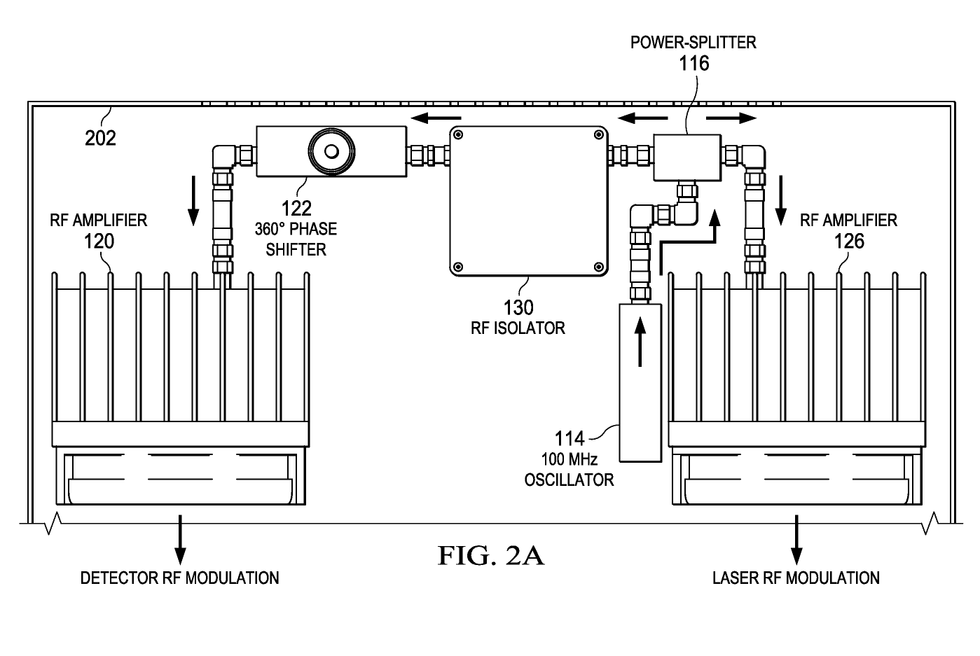

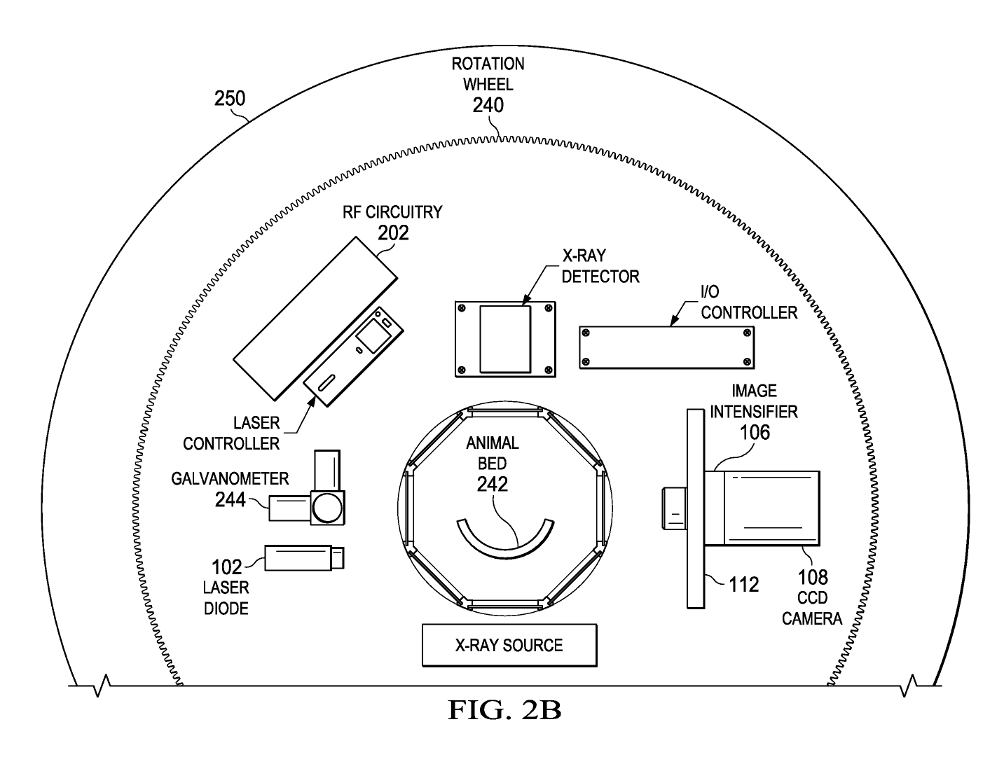

FIG. 2A shows the assembled RF components within an enclosure 202 (e.g., a 14 inch×10 inch×5 inch rectangular box) designed for mounting into a CT gantry. FIG. 2B shows the enclosure 202 and other components of the NIRF FDPM system 100 disposed within a gantry 250. Embodiments of the present disclosure integrate the NIRF FDPM system 100 within a CT gantry. In some embodiments, a CT scanner (e.g., Inveon CT scanner by Siemens, Knoxville, Tenn., USA) is configured to house CT instrumentation and an optional SPECT device on a single rotation wheel 240 which rotates around a horizontally placed animal bed 242. To integrate the FDPM-based NIRF imager with the scanner, the optical and electronic components illustrated in FIG. 1 are mounted in the space reserved for the SPECT instrumentation. In other embodiments, SPECT, NIR, and CT components are housed within the same gantry. An automated filter wheel placed in-front of the ICCD camera selects the appropriate filters 112: e.g., 830 nm bandpass filter assembly for fluorescence imaging and a neutral density filter (OD=7) for white light imaging. The imaging detector or ICCD camera is mounted on a motorized linear stage for translation in the radial direction allowing control over the camera field of view (FOV) without having to change the optical components. A compact, combinational controller (e.g., ITC133, Thorlabs, Newton, N.J.) is used to power the laser diode and to maintain the source or diode temperature.

A dual-axis galvanometer 244 (e.g., MicroMax 673 Series, Cambridge Technology, Lexington, Mass.) mounted near the laser mount 104 or source scans the modulated excitation light beam across the surface of the subject or object. Multifunction data acquisition interfaces (e.g., USB-6009, USB-6216, National Instruments, Austin, Tex.) provide external control and monitoring of the FDPM instruments via, e.g., a USB cable mounted in the CT cable carrier. An FDPM control computer (control and image processing subsystem 124) is also interfaced to the gantry system thus allowing an integrated workflow for CT and FDPM modalities. The image detector may be mounted on the wheel 240 opposite the galvanometer 244 or the light source 102. In some embodiments, an image detector may be mounted on the wheel 240 adjacent to the galvanometer 244 or the light source 102 to allow for measurement of reflectance. Such embodiments may be advantageous for imaging large volume objects that provide a weak transillumination signal.

When docked to the CT scanner, a dedicated PET scanner (Siemens, Knoxville, Tenn., USA) enables sequential CT, FDPM, and PET measurements by automated translation of the animal bed 242 between the dedicated CT and PET gantries.

The control and image processing subsystem 124 includes a processor 134 and instructions executable by the processor 134 to control the optical and RF components and to process images captured by the imaging detector or CCD camera 108. The instructions may be stored in a computer-readable storage device 136 accessed by the processor 134. The control and image processing subsystem 124 may include various other components such as user interfaces (display devices, user input devices, etc.), networking components (wired or wireless network adapters), etc.

The processor 134 may include a general-purpose microprocessor, a digital signal processor, a microcontroller, or other device capable of executing instructions retrieved from a computer-readable storage medium. Processor architectures generally include execution units (e.g., fixed point, floating point, integer, etc.), storage (e.g., registers, memory, etc.), instruction decoding, peripherals (e.g., interrupt controllers, timers, direct memory access controllers, etc.), input/output systems (e.g., serial ports, parallel ports, etc.) and various other components and sub-systems.

The storage 136 is a non-transitory computer-readable storage medium suitable for storing instructions that are retrieved and executed by the processor 134 to perform the functions disclosed herein. The storage 136 may include volatile storage such as random access memory, non-volatile storage (e.g., a hard drive, an optical storage device (e.g., CD or DVD), FLASH storage, read-only-memory), or combinations thereof. The storage 136 also includes control instructions 140 the processor executes to control the operation of the system 100 and other components shown in FIG. 2B or described herein.

The storage 136 includes image processing instructions 138 that the processor 134 executes to produce images from the optical and/or X-ray data generated by or in conjunction with the system 100. For example, to enable image reconstruction, the system 100 executes an algorithm embodied in the image processing instructions 138 that utilizes a 3-D CT-defined volumetric mesh as tomographic input in addition to the multiple 2-D projections of the acquired emission signal, and precise mapping of excitation light distribution onto the 3-D tissue surface.

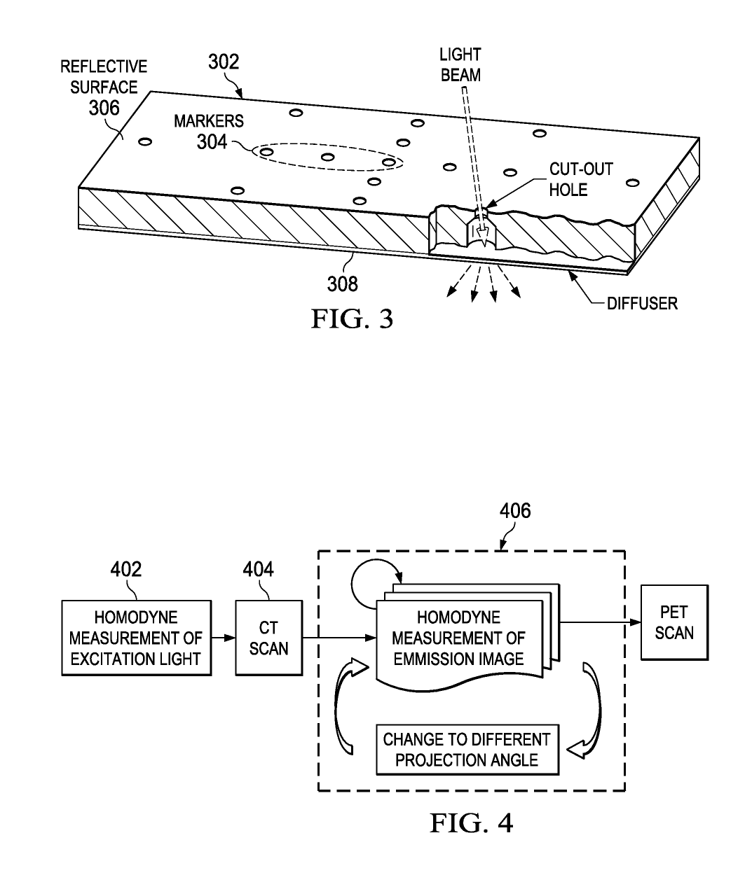

For FDPM measurements conducted within the gantry 250, a transformation matrix is generated, by the processor 134, to map the 2-D FDPM measurements onto the 3-D surface. CT scan (voxel size: cube of 0.11 mm) and 2-D optical images (512×512 pixels) with different FOVs (pixel sizes: squares of 0.2-0.34 mm length) are acquired for a customized phantom 302 made of black plastic (75×32×5 mm) with a highly reflective (e.g. white color) top surface, and multiple drilled holes. FIG. 3 shows a schematic of the phantom 302 with only a few hole/channel openings illustrated for brevity. The 0.6 mm diameter openings of the holes 304 on the reflective surface 306 act as fiducial markers for both CT and optical imaging. The contrast between air and plastic allows easy identification of the openings in the 3-D CT image. The reflective surface 306 and the black plastic 308 exposed through the holes creates high contrast and allows easy identification of the openings in 2-D optical images. The coordinate data of the markers on the reflective surface is measured from CT and 2-D optical imaging. By tracing the coordinate data of the markers in the 2-D images for different FOVs, the distance in the radial direction between the center of the gantry and the optical camera is estimated (e.g., 150-260 mm) with an accuracy of e.g., 1.1 mm standard deviation. This allows estimation of the position of the imaging detector or optical camera 108 relative to the CT FOV and the focal length and detector size.

By matching the coordinate data of all the openings 304 in both modalities, a transformation matrix is created by calculating the location and orientation of the imaging detector or camera 108 relative to the CT gantry coordination system with an estimated error of ±1 pixel (STD) on the CCD. The transformation matrix allows accurate mapping of the 2-D fluorescence intensity distribution from imaging detector or ICCD camera 108 onto the 3-D volumetric mesh obtained from the CT scan. In addition, to register placement of incident excitation light on the phantom surface via the scanning galvanometer 244, optical data is acquired to correlate the galvanometer position with the alignment of the collimated laser beam through the openings. Upon matching the parameters of galvanometer with the 3-D coordinate data of the holes, a formula for tracing the exact ray position for the excitation light distribution on the 3-D surface is obtained.

The protocol associated with PET-CT image co-registration involves sequential CT and PET scanning of a standard cylindrical phantom with four embedded point-sources (Na-22). The offset and orientation for the acquired PET images are then adjusted in order to align them with the CT images. Matching may be accomplished with a translation resolution of 0.1 mm and rotational resolution of 0.1°.

The system 100 acquires FDPM parameters as follows. In order to record the time-dependency, N phase-delays over a complete cycle of 0° to 360° are imposed on the RF signal driving the imaging detector or intensifier 106. For each phase-delay, a steady-state image at the intensifier phosphor screen is captured by the CCD array 108. To account for the ambient, readout, and dark current noise, a baseline image is acquired, by turning off the excitation light, and subtracted from all subsequent phase-sensitive images. The modulation amplitude (I_{Ac}) and phase (θ) are then calculated by performing fast Fourier transform (FFT) on the FDPM measurements.

For data acquisition inside the gantry 250, an object to be imaged (e.g., phantom/mouse) is suspended on a customized bed 242 consisting of thin wires and rods or a heated glass bed, to evenly support the object. This setup allows unimpeded passage of excitation light to the object and collection of emission signals from its surface over several projection angles. The modified animal bed 242 is compatible with all the three imaging modalities.

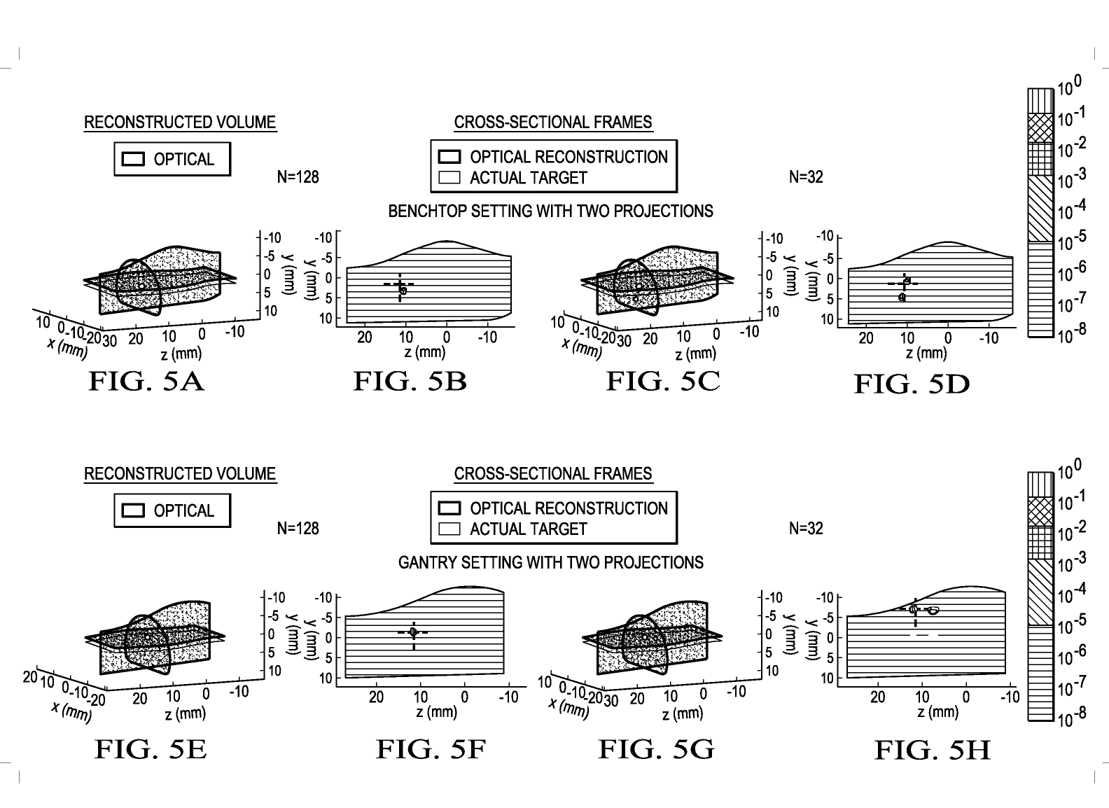

FIG. 4 shows a flow diagram of an overview of a protocol for performing FDPM-based measurements within the gantry. At 402, to account for the implicit phase-delay associated with the involved instrumentation, homodyne detection is first conducted at the excitation wavelength without the intervening object. This baseline phase-delay in each projection is then subtracted from the delay computed from the actual emission signals at the corresponding projections. After baseline phase-delay measurements, the object is moved into the CT FOV for CT scan at 404. Next, for a stationary bed position, homodyne measurements of emission photon distribution are collected at different projection angles at 406. The transformation matrix, as described above, is used for mapping the 2-D optical images onto the surface of 3-D CT-generated object volume.

Embodiments of the system 100 apply time-dependent measurements using a frequency-domain approach. To enable such measurements, embodiments modify a CW-based algorithm for NIRF tomography, derived using high-order simplified spherical harmonics (SP_{N}) approximation to the radiative transfer equation (RTE). As compared to the CW-based algorithm, the measured light density at excitation and emission wavelengths is complex in nature, given by φ^{[x,m]}=I_{AC}^{[x,m]}e^{-jθ[x,m], where [x,m] denotes the variables at excitation ([x]) or emission ([m]) wavelengths, respectively. The tissue absorption coefficient can be expressed as μ}ₐ^{[x,m]}+iω/c_{x,m}, where μₐ^{[x,m]} is the absorption coefficient of the tissue at the excitation/emission wavelengths in CW mode; ω is the modulation frequency; and c_{x,m} is the speed of light at excitation/emission wavelengths within the tissues.

Applying SP₃ approximation along with relevant boundary conditions produces:

[J^{+,m,b}]=[G][μₐ^{[f]}], (1)

where:
G indicates the relationship matrix between J^{+,m,b} and fluorescent absorption distribution μₐ^{[f]};
superscript b represents the variables present only at the tissue surface; and
J^{+,m,b} is the measurable exiting partial current for the emission wavelength and given by

$$\begin{matrix}{J}^{+ , m , b} = {J}_{R}^{+ , m , b} + i {J}_{i}^{+ , m , b} \\ = ( \frac{1}{4} + {J}_{0} ) ( {ϕ}_{1}^{[ m ]} - \frac{2}{3} {ϕ}_{2}^{[ m ]} ) - ( \frac{0.5 + {J}_{1}}{3 {μ}_{a 1}^{[ m ]}} ) v \cdot ∇ {ϕ}_{1}^{[ m ]} + \\ \frac{1}{3} ( \frac{5}{16} + {J}_{2} ) {ϕ}_{2}^{[ m ]} - ( \frac{{J}_{3}}{7 {μ}_{a 3}^{[ m ]}} ) v \cdot ∇ {ϕ}_{2}^{[ m ]} ,\end{matrix} \qquad ( 2 )$$

where:
the coefficients J₀, . . . , J₃ are defined as in Klose A D and Larsen E W 2006, Light transport in biological tissue based on the simplified spherical harmonics equations J. Comput. Phys. 220 441-70, and J_{R}^{+m,b} and J_{I}^{+,m,b} are the real and imaginary parts of J^{+,m,b};
v is the outgoing unit vector normal to the boundary;
φ_{1,2}^{[x,m]} are complex variables and denotes the composite moments of the Legendre moments for excitation and emission radiances;
μ_{a,i}^{[m]} denotes the i-th (i=1, 2, 3) absorption coefficients at emission wavelengths and is equal to μₐ^{[m]}+μₛ^{[m]}(1−gⁱ)+iω/c_{x,m};
μₛ^{[m]} is the tissue scattering coefficient at the emission wavelength; and
g is the anisotropic factor.

Rewriting equation (1), after including complex characteristics, produces:

$$[ \begin{matrix}{J}_{R}^{+ , m , b} \\ {J}_{I}^{+ , m , b}\end{matrix} ] = [ \begin{matrix}{G}_{R} \\ {G}_{I}\end{matrix} ] [ {μ}_{a}^{[ f ]} ] , \qquad ( 3 )$$

where G_{R} and G_{I} are the real and imaginary parts of G. When there are multiple illuminations (Nᵥ) at different positions:

J_{T}^{+,m,b}=Aμₐ^{[f],} (4)

where:

$${J}_{T}^{+ , m , b} = [ \begin{matrix}{J}_{R}^{+ , m , b , 1} \\ {J}_{I}^{+ , m , b , 1} \\ {J}_{R}^{+ , m , b , 2} \\ {J}_{I}^{+ , m , b , 2} \\ … \\ {J}_{R}^{+ , m , b , {N}_{v}} \\ {J}_{I}^{+ , m , b , {N}_{v}}\end{matrix} ] A = [ \begin{matrix}{G}_{R}^{1} \\ {G}_{I}^{1} \\ {G}_{R}^{2} \\ {G}_{I}^{2} \\ … \\ {G}_{R}^{{N}_{v}} \\ {G}_{I}^{{N}_{v}}\end{matrix} ] . \qquad ( 5 )$$

Embodiments employ limited memory variable metric-bound constrained quasi-Newton method to solve the following least squares problem for fluorescence recovery:

min θ(μₐ^{[f]}):∥Aμₐ^{[f]}−J_{T}^{+,m,b}∥² subject to 0<μₐ^{[f]}<μₐ^{f,sup}, (6)

where μₐ^{f,sup} is the upper constraint on μₐ^{[f]}.

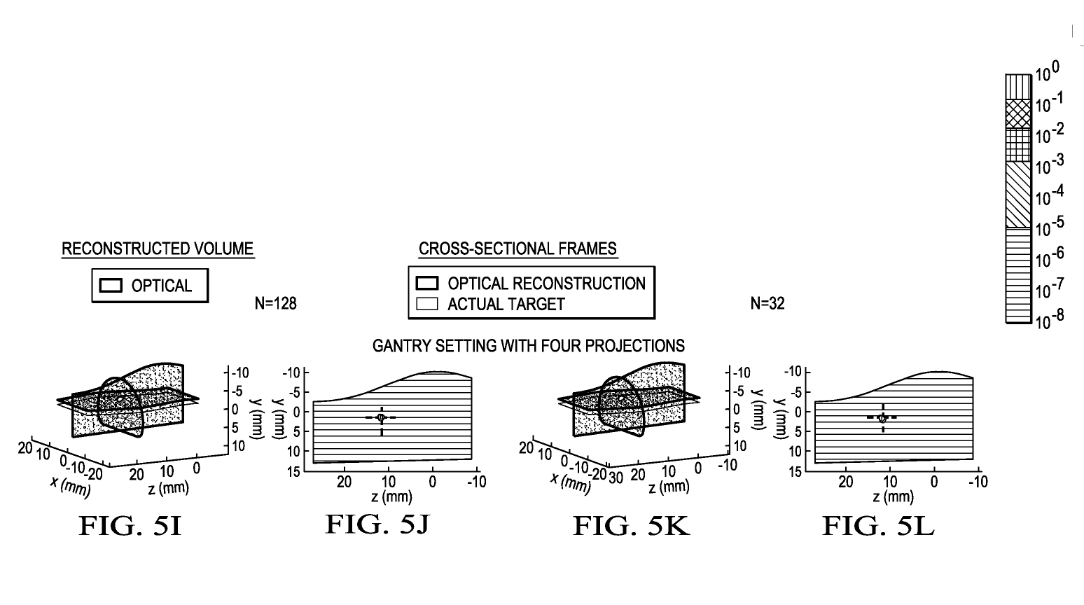

FIGS. 5A-5L show exemplary tomographic reconstructions using: (1) a bench top system (1^{st} row, FIGS. 5A-5D), and a gantry installed system 100 with (2) 2-projections (2^{nd} row, FIGS. 5E-5H) and (3) 4-projections (3^{rd} row, FIGS. 5I-5L). For brevity, reconstructions with only N=128 and N=32 phase-delays are included. The 3-D figures highlight the fluorophore localization within the reconstructed volume. Target localization errors are represented by the cross sectional frames with thin and thick boundaries for 3D figures indicating the center position of the actual (CT derived) and optically reconstructed target, respectively. The volumetric mesh denotes the top 80% of the contour levels for the reconstructed fluorophore distribution. 2D slices show logarithmic intensity maps of the fluorophore along with the artifacts generated internal to the reconstructed volume. The cross-hairs on the 2D plots indicate the actual position of the fluorophore.

An embodiment of the system 100 can be applied to perform fluorescence gene reporter tomography (FGRT). Emission tomography makes use of the surface measurement of emitted light for mathematical reconstruction of the source of light emitting gene reporter. Compared to bioluminescence tomography (BLT), FGRT may provide more facile and robust 3-D image reconstructions due to potentially higher photon count rate, ability to conduct time-dependent measurements, as well as the possible combinations of multiple incident excitation patterns with multiple projection measurements of emitted light. The system 100 provides for acquisition of multiple projections via the rotating gantry-based imaging system, and also allows for integration of other imaging modalities such as nuclear and X-ray computed tomography.

In an embodiment of the system 100 configured for FGRT, the laser diode 102 is selected for operation in the 690 nm range. A 690 nm bandpass filter is used to ensure the monochromatic light modulation of the laser diode 102. The collected light is passed through a 720 nm filter before incident on the image intensifier 106.

In a method for FGRT, an embodiment of the system 100 configured for FGRT is employed in conjunction with a linear regularization-free reconstruction algorithm employing the third-order simplified harmonics spherical approximation (SP₃) to the radiative transfer equation (RTE) and a 3D volume mesh obtained from CT. Some embodiments may also apply a priori anatomical information obtained from CT. Using the system 100, multiple projection images may be acquired from transillumination of an point excitation light as the gantry is rotated (e.g., 0, 45, 180, and 315 degrees). The fluorescent photon distribution may be mapped onto the surfaces defined by CT.

In order to perform FGRT, embodiments employ a linear regularization-free reconstruction strategy developed by neglecting the absorption coefficient of the fluorescence gene reporter at the excitation wavelength. In other words, the attenuation of excitation light from the gene reporter was assumed to be small compared to that from endogenous chromophores. Based on this assumption, the third-order SP₃ approximation achieves more accurate reconstruction quality when compared to the classic diffusion approximation (DA) because a more precise solution to the forward problem of photon propagation is obtained from the SP₃. Briefly, the linear regularization-free reconstruction method was developed by using the emission equation of the SP₃:

$$[ \begin{matrix}{M}_{1 {ϕ}_{1}^{m}} & {M}_{1 {ϕ}_{2}^{m}} \\ {M}_{2 {ϕ}_{1}^{m}} & {M}_{2 {ϕ}_{2}^{m}}\end{matrix} ] [ \begin{matrix}{ϕ}_{1}^{m} \\ {ϕ}_{2}^{m}\end{matrix} ] = [ \begin{matrix}{B}^{m} & \\ & - \frac{2}{3} {B}^{m}\end{matrix} ] [ {μ}_{a}^{sf} ] \qquad ( 7 )$$

where M_{iφ}_{ⱼ}_{^{m}} is the submatrix corresponding to φⱼ^{m} (the composite moments of the radiance) in the i-th SP₃ equation by using the finite element methods and B^{m} is obtained by its components b_{pq}^{m} and given as

b_{pq}^{m}=∫_{Ω}Qφ^{x}vₚ·v_{q}dr (8)

where:
Ω is the domain for reconstruction;
r is the location in Ω;
v_{p,q} are the shape functions;
Q is the quantum efficiency of the fluorescence gene reporter; and
φ^{x} is the excitation fluence and is obtained by directly solving the SP₃ excitation equation.

Inverting the matrix on the left-hand side of equation (7) produces:

$$\{ \begin{matrix}{ϕ}_{1}^{m} = ( {IM}_{1 {ϕ}_{1}^{m}} - \frac{2}{3} {IM}_{1 {ϕ}_{2}^{m}} ) {B}^{m} {μ}_{a}^{sf} \\ {ϕ}_{2}^{m} = ( {IM}_{2 {ϕ}_{1}^{m}} - \frac{2}{3} {IM}_{2 {ϕ}_{2}^{m}} ) {B}^{m} {μ}_{a}^{sf}\end{matrix} .$$

Removing the rows in matrices (IM_{1φ}_{₁}_{^{m}}−⅔IM_{1φ}_{₂}_{^{m}})B^{m} and (IM_{2φ}_{₁}_{^{m}}−⅔IM_{2φ}_{₂}_{^{m}})B^{m} corresponding to the non-boundary measurable discretized points, and applying the exiting partial current equation,

$${J}^{+ , m , b} = ( \frac{1}{4} + {J}_{0} ) ( {ϕ}_{1}^{m} - \frac{2}{3} {ϕ}_{2}^{m} ) - ( \frac{0.5 + {J}_{1}}{3 {μ}_{a 1}^{m}} ) v \cdot ∇ {ϕ}_{1}^{m} + \frac{1}{3} ( \frac{5}{16} + {J}_{2} ) {ϕ}_{2}^{m} - ( \frac{{J}_{3}}{7 {μ}_{a 3}^{m}} ) v \cdot ∇ {ϕ}_{2}^{m} \qquad ( 9 )$$

produces:

J^{+,m,b}=Gμₐ^{sf} (10)

where:
coefficients J₀, . . . , J₃ are as in Klose, A. D. and E. W. Larsen, Light transport in biological tissue based on the simplified spherical harmonics equations. Journal of Computational Physics, 2006. 220(1): p. 441-470; and
G is the relationship matrix between the unknown μₐ^{sf} and the acquirable measurements J^{+,m,b}.

With Nᵥ different illuminations at different positions:

J_{T}^{+,m,b}=Aμₐ^{sf} (11)

where:

J_{T}^{+,m,b}=[J₁^{+,m,b}, . . . , Jₙ_{ᵥ}^{+,m,b}, . . . , J_{N}_{ᵥ}^{+,m,b}]^{T},

A=[G₁, . . . , Gₙ_{ᵥ}, . . . , G_{N}_{ᵥ}]^{T}, and
T is a transpose operator.

Finally, the limited memory variable metric-bound constrained quasi-Newton method (BLMVM) may be applied to solve the following least squares problem for the linear regularization-free FGRT:

$${min}_{0 < {μ}_{a}^{sf} < {μ}_{a}^{sf , sup}} θ ( {μ}_{a}^{sf} ) \text{:} {‖ A {μ}_{a}^{sf} - {J}_{T}^{+ , m , b} ‖}^{2}$$

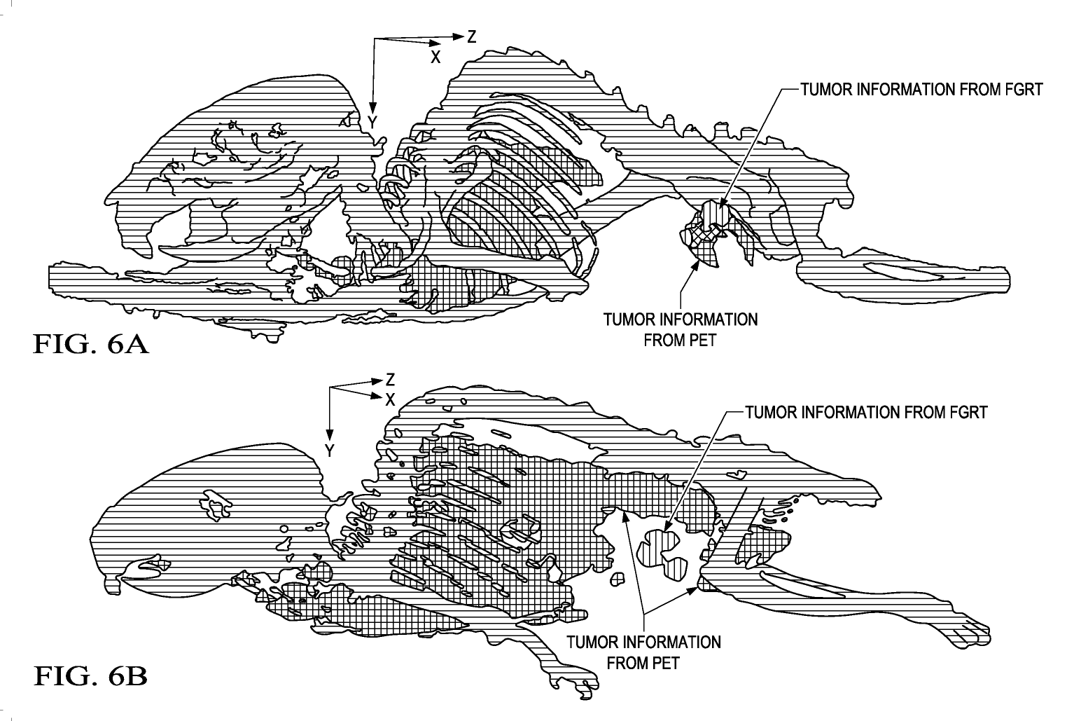

The control and image processing subsystem 124 may apply the algorithm to reconstruct an imaged region of a subject or object using, for example, tetrahedral volumetric meshing. FIGS. 6A and 6B show multimodal reconstructed results for mice imaged 4 weeks and 11 weeks, respectively, after implantation of human prostate cancer cells. Tumor contours obtained from FGRT, the skeleton contours obtained from CT, and PET signal from the radiolabeled antibody are shown. As expected with antibody imaging, clearance occurs through the liver, hence the PET signal within the abdomen. When the tumor is in its early stage, the FGRT reconstructed results agree well with PET imaging.

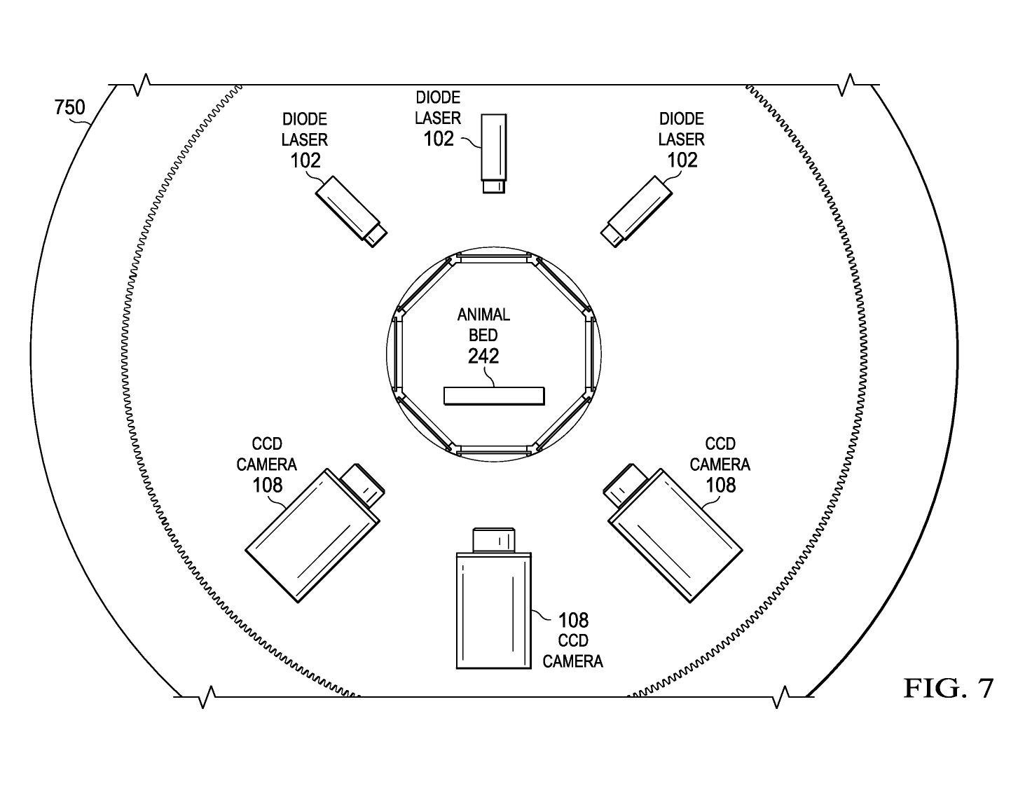

An alternative embodiment of a NIRF imaging system 100 used for CW and FDPM measurements is shown in FIG. 7. Rather than mounting a single laser and a single camera on a rotation wheel to provide imaging from a number of projection angles, a plurality of lasers (e.g., instances of laser diode 102) and a plurality of imaging detectors or cameras (e.g., instances of the camera 108) are positioned on a stationary gantry 750 at fixed locations about a bed (e.g., bed 242) for imaging of an object located on the bed from a plurality of angles. For example, each laser 102 and corresponding camera 108 may be activated in sequence to image the object. Each of the plurality of lasers 102 and cameras 108 may operate as described above with respect to the system 100, and RF circuitry and CW components as shown in and described with regard to FIG. 1.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. An optical tomography system, comprising:
a bed configured to support an object to be imaged;
a wheel configured to rotate about the bed;
a light source coupled to the wheel;
an image detector coupled to the wheel and disposed to capture images of the object;
radio frequency (RF) circuitry coupled to the light source and the image detector, the radio frequency circuitry configured to simultaneously:
generate a modulation signal to modulate the light source; and
generate a demodulation signal to modulate a gain of the image detector.

2. The system of claim 1, wherein the RF circuitry comprises:
an oscillator configured to generate an oscillation signal;
a splitter coupled to an output of the oscillator; wherein the splitter is configured to provide the oscillation signal to the light source and the image detector.

3. The system of claim 2, wherein the RF circuitry comprises a phase shifter coupled to the splitter and the image detector, wherein the phase shifter is configured to selectably vary the phase of the oscillation signal provided to the image detector with respect to the light source.

4. The system of claim 2 further comprising, a bias circuit coupled to the light source, the bias circuit configured to superimpose the oscillation signal on a bias voltage that drives the light source.

5. The system of claim 1, wherein the image detector comprises:
a camera coupled to an image intensifier configured to intensify detected fluorescent light; or
a high sensitivity camera that supports gain modulation at a high frequency; and
wherein the demodulation signal modulates the gain of the image detector.

6. The system of claim 5, further comprising a bias circuit coupled to the image detector, the bias circuit configured to superimpose the oscillation signal on a bias voltage that drives the gain of the image detector.

7. The system of claim 1, further comprising a computerized tomography scanner coupled to the wheel.

8. The system of claim 1, further comprising a plurality of optical filters disposed on the wheel between the bed and the image detector.

9. The system of claim 1, further comprising a motorized mount coupling the image detector to the wheel, wherein the motorized mount is configured to vary the distance between the image detector and the object.

10. The system of claim 1, further comprising a control and image processing system configured to:
generate a transformation matrix to map frequency domain photon migration (FDPM) and continuous wave (CW) measurements generated from images acquired by the image detector to computerized tomography (CT) scans that define the boundary surface; wherein the matrix is generated by relating spatial coordinates of a phantom surface collected using optical measurements (FDPM or CW) to spatial coordinates derived from CT scans of the phantom surface;
apply the transformation matrix to generate a boundary position of FDPM measurements for use in generating an interior image of fluorescence.

11. A method for performing frequency domain photon migration (FDPM) and continuous wave (CW) measurements in an optical tomography system, comprising:
generating, by the optical tomography system, a transformation matrix that allows determination of surface location of fluorescence measurements and excitation illumination locations using a calibration phantom to determine boundary surface locations defined by a computerized tomography (CT) scanner;
positioning an object to be imaged in a path between a light source and an image detector;
illuminating the object with the light source from a plurality of angles and capturing fluorescence data produced by the object responsive to the illuminating;
irradiating the object with X-rays and capturing the X-rays to generate a CT scan of the boundary surface;
applying the transformation matrix to align captured fluorescence data with surface locations that correspond to the surfaces acquired from the CT scan; and
generating a composite image comprising the aligned fluorescence data and the CT scan image.

12. The method of claim 11, further comprising:
acquiring a baseline measurement of phase delay in optical signal generation and capture paths of the optical tomography system by measuring emission of a modulated light source via an image detector comprising a modulated gain;
positioning a calibration phantom in a path between the light source and the image detector;
defining a surface of the calibration phantom relative to the light source and relative to the CT scanner to generate the transformation matrix.

13. The method of claim 12, further comprising determining a position of a galvanometer mirror to illuminate different surface locations on the calibration phantom.

14. The method of claim 11, further comprising:
modulating the light source with a bias voltage applied to a radio frequency (RF) oscillating signal;
modulating gain of the image detector with a bias voltage applied to an RF oscillating signal.

15. The method of claim 14, further comprising shifting phase of the oscillating signal applied to the image detector relative to the oscillating signal applied to the light source by up to 360 degrees as the object is illuminated by the light source at the plurality of angles.

16. The method of claim 11, wherein illuminating the object with the light source comprises rotating a wheel to which the light source and image detector are attached to position the light source and image detector at the plurality of angles.

17. An optical tomography system, comprising:
a bed configured to support an object to be imaged;
a plurality of light sources disposed about the bed and configured to illuminate the object from different angles;
a plurality of image detectors disposed about the bed and configured to capture images of the object as the object is illuminated by the light sources;
radio frequency (RF) circuitry coupled to the light sources and detectors, the RF circuitry configured to:
modulate the light sources; and
demodulate image signals detected by the image detectors.

18. The system of claim 17, wherein the RF circuitry comprises:
an oscillator configured to generate an oscillation signal;
a splitter coupled to an output of the oscillator; wherein the splitter is configured to provide the oscillation signal to the light sources and the image detectors.

19. The system of claim 18, wherein the RF circuitry comprises a phase shifter coupled to the splitter and the image detectors, wherein the phase shifter is configured to selectably vary the phase of the oscillation signal provided to the image detectors with respect to the light sources.

20. The system of claim 18, wherein each of the image detectors comprises a camera coupled to an image intensifier configured to intensify detected light, and wherein the phase varied oscillation signal provided to the image detectors modulates a gain of the image detector.
